(19) United States
(12) Patent Application Publication
(10) Pub. No.: US 2021/0290513 A1
(43) Pub. Date: Sep. 23, 2021

(54) COMPOSITION COMPRISING KAKKALIDE

(71) Applicant: LG HOUSEHOLD & HEALTH CARE LTD., Seoul (KR)

(72) Inventors: YoungJe AHN, Seoul (KR); So Young LEE, Seoul (KR); Mu Hyun JIN, Seoul (KR)

(73) Assignee: LG HOUSEHOLD & HEALTH CARE LTD., Seoul (KR)

(21) Appl. No.: 17/205,188

(22) Filed: Mar. 18, 2021

(30) Foreign Application Priority Data
Mar. 19, 2020 (KR) 10-2020-0033765

(51) Int. Cl. A61K 8/60; A61Q 19/02; A61Q 19/08; A61Q 7/00; A61Q 5/00

(52) U.S. Cl. CPC A61K 8/60; A61Q 19/02; A61K 2800/92; A61Q 7/00; A61Q 5/006; A61Q 19/08

(57) ABSTRACT

The present invention relates to a composition comprising kakkalide. In addition, the composition of the present invention comprising kakkalide can be variously used for brightening the skin, improving wrinkles in the skin, moisturizing the skin, inhibiting bacteria in the skin, preventing hair loss, or promoting hair growth.

### CROSS-REFERENCE TO RELATED APPLICATION

This application claims priority to and the benefit of Korean Patent Application No. 2020-0033765, filed on Mar. 19, 2020, the disclosure of which is incorporated herein by reference in its entirety.

### BACKGROUND

### 1. Field of the Invention

The present invention relates to a composition for brightening the skin, improving wrinkles, moisturizing, inhibiting bacteria, preventing hair loss, or promoting hair growth, which comprises kakkalide.

### 2. Discussion of Related Art

Puerariae Flos, or Gal Flower in Korean, refers to a flower bud of Pueraria lobata or a Pueraria lobata flower just beginning to bloom. Puerariae Flos has an uneven, long oval or bell-like shape, a length of 5 to 15 mm, a width of 2 to 6 mm, and a thickness of 2 to 3 mm. The calyx is dark green and joined at the base, and the end is split into five lobes, two of which are pointed and look as if they are joined together. The outer surface is covered with yellowish white hairs. When viewed through a magnifying glass, there are five petals, which are bluish purple or purplish brown and are wrapped in the calyx. There are 10 stamens, and the pistils are thin, long, bent, and have hairs attached thereto (Non-Patent Document 1).

Aging is a phenomenon of life, and all living things begin to age from the moment they are born. Due to the improvement of hygiene, health, and the environment, a human lifespan has been significantly extended from in the past, and the proportion of the elderly population is steadily increasing (Patent Document 1). In addition, as the lifespan of humans has increased, public awareness of the quality of life has changed and interest in health has increased, so research on the development of new materials based on natural products/resources has been actively conducted. Expectations for natural products (especially plant resources) that have relatively few side effects compared to synthetic drugs with possible side effects are gradually increasing (Non-Patent Document 2).

In the above-described background of aging and longevity, beauty care provides a sense of satisfaction and happiness by supplementing shortcomings in an individual's appearance, and thereby helps to easily maintain and develop social interactions and, further, contributes to the promotion of social health as well as the promotion of individual mental health (Non-Patent document 3). All humans, regardless of age, have an instinct to pursue beauty, and they become more interested in their appearance as they age.

Under this background, the inventors of the present invention have researched and developed a composition that has fewer side effects and is effective for brightening the skin, improving wrinkles, moisturizing, inhibiting bacteria, preventing hair loss, or promoting hair growth.

### RELATED-ART DOCUMENTS

### Patent Document

(Patent Document 0001) KR 10-2015-0180221

### Non-Patent Documents

(Non-Patent Document 0001) The Korean Herbal Pharmacopoeia, Korea Food and Drug Administration, Jan. 29, 2013.

(Non-Patent Document 0002) Drug Development Using Natural Materials, Biotech Policy Research Center, Dec. 3, 2010.

(Non-Patent Document 0003) The Effects of Middle and Elderly Woman's Skin Care on Happiness. Youn, Kim, Kor. J. Aesthet. Cosmetol., 8: 1-13, 2010.

### SUMMARY OF THE INVENTION

As a result of making efforts to discover a composition having the ability to brighten the skin, improve wrinkles, moisturize, inhibit bacteria, prevent hair loss, or promote hair growth, the inventors of the present invention discovered that kakkalide has the ability to brighten the skin, improve wrinkles, moisturize, inhibit bacteria, prevent hair loss, or promote hair growth, and thus completed the present invention.

The present invention is directed to providing a composition for brightening the skin, improving wrinkles, moisturizing, inhibiting bacteria, preventing hair loss, or promoting hair growth, which comprises kakkalide as an active ingredient.

The composition may be a cosmetic composition, a pharmaceutical composition, or a food composition.

In addition, the present invention is directed to providing a method of brightening the skin, improving wrinkles, moisturizing, inhibiting bacteria, preventing hair loss, or promoting hair growth, which comprises treating a subject with kakkalide.

In addition, the present invention is directed to providing a method of preventing or treating a disease related to excessive pigmentation, which comprises treating a subject with kakkalide.

In addition, the present invention is directed to providing a method of preventing or treating symptoms of dry skin, which comprises moisturizing the skin by treating a subject with kakkalide.

In addition, the present invention is directed to providing a method of preventing or treating dandruff, which comprises treating a subject with kakkalide.

One aspect of the present invention provides a composition comprising kakkalide.

Another aspect of the present invention provides a method of brightening the skin, improving wrinkles, moisturizing, inhibiting bacteria, preventing hair loss, or promoting hair growth, which comprises treating a subject with kakkalide.

In the present invention, kakkalide may be used alone or in the form of a composition comprising kakkalide and a carrier, and the composition may have a form that comprises an additional component within a range that does not impair effects of the kakkalide of the present invention. Depending on the purpose of use, the carrier may be selected from among a cosmetically acceptable carrier, a pharmaceutically acceptable carrier, or a sitologically acceptable carrier. The additional component used in the composition comprises an ingredient legally permitted by the government of the relevant country.

In the present invention, the treatment refers to applying kakkalide or a composition comprising the same to the skin or administering kakkalide or a composition comprising the same to a subject. The administration comprises both the oral and parenteral administration of kakkalide or a composition comprising the same to a subject.

In the present invention, “brightening” means not only brightening skin tone, but also preventing, alleviating, treating, or improving diseases related to excessive pigmentation which are caused by ultraviolet rays, hormones or genetics. These diseases related to excessive pigmentation have symptoms that are caused by excessive deposition of skin pigment, and may comprise melasma, freckles, or hyperpigmentation.

According to one embodiment of the present invention, it was confirmed that kakkalide has the effect of reducing the amount of melanin at the cellular level (Experimental Example 1).

Therefore, the composition of the present invention may be a composition for skin brightening comprising kakkalide as an active ingredient.

In addition, in the present invention, a method of brightening the skin comprising treating a subject with kakkalide is provided.

In addition, in the present invention, a method of preventing or treating a disease related to excessive pigmentation is provided.

In the present invention, “improving wrinkles” means preventing, inhibiting, or suppressing the occurrence of wrinkles in the skin, or alleviating wrinkles that have already occurred.

According to one embodiment of the present invention, it was confirmed that kakkalide has the effect of promoting the synthesis of type I collagen in human-derived fibroblasts (Experimental Example 2).

Therefore, the composition of the present invention may be a composition for improving wrinkles in the skin comprising kakkalide as an active ingredient.

In addition, in the present invention, a method of preventing or improving wrinkles in the skin comprising treating a subject with kakkalide is provided.

In the present invention, “moisturizing” means supplying moisture to the skin that has become dry, rough, and flaky due to dry air and irritation, preventing evaporation of moisture to maintain the skin's flexibility, and inducing uniform exfoliation of dead skin cells to maintain a smooth surface. In addition, in the present invention, “moisturizing” means preventing, alleviating, treating, or improving symptoms of dry skin. The symptoms of dry skin may comprise lack of moisture in the skin, atopic dermatitis, psoriasis, xeroderma, eczema, or xeroderma pigmentosum.

According to one embodiment of the present invention, it was confirmed that kakkalide has the effect of inducing hyaluronic acid synthesis, considering that kakkalide increases HAS2, HAS3, and AQP3 mRNA expression in human keratinocytes (Experimental Example 3).

Therefore, the composition of the present invention may be a composition for moisturizing the skin comprising kakkalide as an active ingredient.

In addition, in the present invention, a method of moisturizing the skin comprising treating a subject with kakkalide is provided.

In addition, in the present invention, a method of preventing, improving, or treating symptoms of dry skin comprising treating a subject with kakkalide is provided.

In the present invention, “inhibiting bacteria (antibacterial)” can be used interchangeably with “inhibiting microbes (antimicrobial)” or “inhibiting dandruff-causing fungi (antifungal against dandruff-causing fungi)” and means inhibiting the proliferation of bacteria or killing bacteria, and comprises all mechanisms taking place to provide protection against the action of microorganisms such as bacteria and fungi. In addition, in the present invention, “inhibiting bacteria (antibacterial)” may mean preventing, alleviating, treating, or improving dandruff by inhibiting the growth of Pityrosporum ovale.

According to one embodiment of the present invention, it was confirmed that kakkalide has an antibacterial effect against Pityrosporum ovale, which is a dandruff-causing fungus (Experimental Example 4).

Therefore, the composition of the present invention may be a composition for inhibiting bacteria comprising kakkalide as an active ingredient.

In addition, in the present invention, a method of inhibiting bacteria in the skin comprising treating a subject with kakkalide is provided.

In addition, in the present invention, a method of preventing or treating dandruff comprising treating a subject with kakkalide is provided.

In the present invention, “preventing hair loss” means improving hair loss, and may mean delaying the speed of hair loss, reducing the speed or amount of hair falling out, or preventing the thinning of hair. According to the present invention, by using kakkalide, it is possible to improve the condition where hair has been lost, from an area where the hair should normally be present, due to various habits and environmental influences such as genetic causes, hormonal imbalance, mental stress, exposure to air pollution, and consumption of processed food, prevent the progression of hair loss, and induce hair growth.

According to one embodiment of the present invention, it was confirmed that kakkalide has the effect of activating the Wnt/β-catenin signaling pathway, which decreases in the process of hair loss (Experimental Example 5). In addition, in one embodiment of the present invention, it was confirmed that kakkalide has the effect of proliferating dermal papilla cells playing a key role in hair formation by receiving nutrients from surrounding blood vessels (Experimental Example 6).

The hair loss may be, for example, one or more selected from the group consisting of dystrophic hair loss, endocrine disorder-related hair loss, vascular disorder-related hair loss, middle-age hair loss, traction alopecia, alopecia areata, neurogenic alopecia, dandruff-induced hair loss, trichotillomania, malignant hair loss, female pattern hair loss, male pattern hair loss, androgenetic alopecia, telogen effluvium, Tinea capitis, frontal alopecia, hypotrichosis, hereditary hypotrichosis simplex, alopecia universalis, mechanical alopecia, traumatic alopecia, pressure-induced alopecia, anagen effluvium, dandruff-induced hair loss, alopecia syphilitica, seborrheic alopecia, symptomatic hair loss, alopecia cicatrisata, and congenital alopecia, but the present invention is not limited thereto.

In addition, in the present invention, “promoting hair growth” means increasing the thickness of produced hair, increasing speed of hair growth, or promoting hair growth.

According to one embodiment of the present invention, it was confirmed that kakkalide has the effect of proliferating dermal papilla cells playing a key role in hair formation by receiving nutrients from surrounding blood vessels (Experimental Example 6).

Therefore, the composition of the present invention may be a composition for preventing hair loss or promoting hair growth comprising kakkalide as an active ingredient.

In addition, in the present invention, a method of preventing hair loss or promoting hair growth comprising treating a subject with kakkalide is provided.

Therefore, in the present invention, a composition comprising a compound represented by the following Chemical Formula 1 is provided.

[Chemical Formula 1]

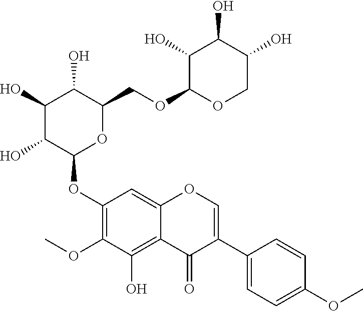 [SMILES: COc1ccc(-c2coc3cc(O[C@@H]4O[C@H](CO[C@@H]5OC[C@@H](O)[C@H](O)[C@H]5O)[C@@H](O)[C@H](O)[C@H]4O)c(OC)c(O)c3c2=O)cc1]

The compound has a molecular formula C₂₈H₃₂O₁₅ and a molecular weight of 608.54, and is named kakkalide (CAS No. 58274-56-9).

In the present invention, a method of obtaining the compound is not particularly limited, and the compound may be extracted and/or purified from natural sources or chemically synthesized by a method known in the art, or a commercially available material may be used.

According to one embodiment of the present invention, the kakkalide of the present invention may be an isoflavonoid-based material extracted from a flower bud of Pueraria lobata or a Pueraria lobata flower just beginning to bloom.

In a cosmetic composition, pharmaceutical composition, or food composition of the present invention, the content of kakkalide is preferably 0.00001 to 10% by weight based on the total weight of the cosmetic composition, pharmaceutical composition, or food composition.

The kakkalide of the present invention may be present in the form of a hydrate or a solvate.

The kakkalide of the present invention may be present in a crystalline or amorphous form, and all these physical forms are comprised in the scope of the present invention.

The composition of the present invention may be prepared in a formulation selected from the group consisting of a solution, an ointment for external use, a cream, a foam, a nourishing toner, an emollient toner, a pack, a softening water, a milky lotion, a makeup base, a serum, a cleanser, a bathing agent, a sunscreen cream, a sun oil, a suspension, an emulsion, a paste, a gel, a lotion, a powder, a soap, a surfactant-containing cleansing product, an oil, a powder-type foundation, an emulsion-type foundation, a wax-type foundation, a patch, and a spray and is preferably a toner, a serum, a lotion, a cream, a pack, a gel, a powder, a foundation, or a cleanser, but the present invention is not limited thereto.

In the present invention, a cosmetic composition for brightening the skin, improving wrinkles in the skin, moisturizing the skin, inhibiting bacteria in the skin, preventing hair loss, or promoting hair growth comprising kakkalide represented by Chemical Formula 1 as an active ingredient is provided.

The cosmetic composition may additionally comprise one or more types of cosmetically acceptable carriers used in general skin cosmetics, and conventional ingredients such as an oil, water, a surfactant, a moisturizer, a lower alcohol, a thickening agent, a chelating agent, a pigment, a preservative, a perfume, and the like may be appropriately used, but the present invention is not limited thereto.

When the formulation of the present invention is a powder or a spray, lactose, talc, silica, aluminum hydroxide, calcium silicate, a polyamide powder, or a combination thereof may be used as a carrier component. In particular, when the formulation of the present invention is a spray, a propellant such as chlorofluorohydrocarbon, propane/butane, or dimethyl ether may be additionally added.

When the formulation of the present invention is a solution or an emulsion, a solvent, a solubilizing agent, or an emulsifying agent is used as a carrier component. Examples thereof comprise water, ethanol, isopropanol, ethyl carbonate, ethyl acetate, benzyl benzoate, propylene glycol, and 1,3-butylglycol oil, and particularly, cottonseed oil, peanut oil, corn germ oil, olive oil, castor oil, sesame oil, a glycerol fatty ester, and a fatty acid ester of polyethylene glycol or sorbitan.

When the formulation of the present invention is a suspension, a liquid diluent such as water, ethanol, or propylene glycol, an ethoxylated isostearyl alcohol, a suspending agent such as polyoxyethylene sorbitol ester or polyoxyethylene sorbitan ester, microcrystalline cellulose, aluminum metahydroxide, bentonite, agar, tragacanth, or the like may be used as a carrier component.

When the formulation of the present invention is a soap, an alkali metal salt of a fatty acid, a fatty acid hemiester salt, a fatty acid-protein hydrolysate, an isethionate, a lanolin derivative, an aliphatic alcohol, a vegetable oil, glycerol, a sugar, or the like may be used as a carrier component.

In addition, in the present invention, a method of brightening the skin, improving wrinkles, moisturizing the skin, inhibiting bacteria, preventing hair loss, or promoting hair growth comprising applying, to the skin of an individual, the composition comprising kakkalide represented by Chemical Formula 1 as an active ingredient is provided. The individual comprises mammals comprising humans, livestock, rats, and the like, but the present invention is not limited thereto.

According to one embodiment of the present invention, a pharmaceutical composition for brightening the skin, improving wrinkles in the skin, moisturizing the skin, inhibiting bacteria in the skin, preventing hair loss, or promoting hair growth comprising kakkalide represented by Chemical Formula 1 is provided.

In the present invention, the term “pharmaceutical composition” may be used as a concept comprising the meaning of “quasi-drug” or “drug.”

The composition comprising the compound of the present invention may be any one of various oral or parenteral formulations and is preferably a parenteral preparation. The preparation is formulated using a diluent or an excipient such as a filler, an extender, a binder, a wetting agent, a disintegrant, a surfactant, or the like that is commonly used. Examples of solid preparations for oral administration comprise tablets, pills, powders, granules, capsules, and the like, and such solid preparations are prepared by adding at least one excipient such as starch, calcium carbonate, sucrose, lactose, gelatin, or the like to one or more compounds. In addition, in addition to the simple excipients, lubricants such as magnesium stearate and talc are also used. Liquid preparations for oral administration comprise suspensions, solutions for internal use, emulsions, syrups, and the like, and may comprise commonly-used simple diluents such as water and liquid paraffin and various excipients such as wetting agents, sweeteners, fragrances, preservatives, and the like. Preparations for parenteral administration comprise sterile aqueous solutions, non-aqueous solutions, suspensions, and emulsions. As a non-aqueous solvent or suspension solvent, propylene glycol, polyethylene glycol, a vegetable oil such as olive oil, an injectable ester such as ethyl oleate, or the like may be used.

In addition, in the present invention, a composition for external use for brightening the skin, improving wrinkles in the skin, moisturizing the skin, inhibiting bacteria in the skin, preventing hair loss, or promoting hair growth comprising kakkalide represented by Chemical Formula 1 as an active ingredient is provided.

When the compound of Chemical Formula 1 is used in a formulation for external use on the skin, adjuvants commonly used in the field of dermatology, such as fatty substances, organic solvents, solubilizers, thickening and gelling agents, emollients, antioxidants, suspending agents, stabilizers, foaming agents, fragrances, surfactants, water, ionic or nonionic emulsifiers, fillers, sequestering and chelating agents, preservatives, vitamins, blockers, wetting agents, essential oils, dyes, pigments, hydrophilic or lipophilic active agents, lipid vesicles, or miscellaneous ingredients commonly used in compositions for external use on the skin, may be additionally comprised. In addition, these ingredients may be introduced in amounts generally used in the field of dermatology.

When the compound of Formula 1 is provided in the form of a composition for external use, the composition may be in an ointment, patch, gel, cream, or spray formulation, but the present invention is not limited thereto.

The composition for external use of the present invention is particularly preferably used as a parenteral preparation. For example, the composition for external use may be prepared by a method of preparing a conventional composition for external use on the skin comprising homogeneously mixing a suitable, pharmaceutically acceptable base such as Vaseline or stearyl alcohol, a suitable, pharmaceutically acceptable surfactant such as polysorbate or sorbitan sesquioleate, a suitable, pharmaceutically acceptable moisturizer such as glycerin, a suitable pharmaceutically acceptable solvent, a flavoring agent, a colorant, a stabilizer, a thickener, and the like.

In addition, in the present invention, a quasi-drug composition for brightening the skin, improving wrinkles in the skin, moisturizing the skin, inhibiting bacteria in the skin, preventing hair loss, or promoting hair growth comprising kakkalide represented by Chemical Formula 1 as an active ingredient is provided.

When the compound of Chemical Formula 1 is provided in the form of a quasi-drug composition, the quasi-drug composition may additionally comprise a pharmaceutically acceptable carrier, excipient, or diluent as necessary, in addition to comprising one or more compounds selected from the group consisting of hydroxycinnamic acid, isoamyl acetate, and betaine or a pharmaceutically acceptable salt thereof as an active ingredient. The pharmaceutically acceptable carrier, excipient, or diluent is not limited as long as it does not impair the effects of the present invention, and examples thereof may comprise fillers, extenders, binders, wetting agents, disintegrants, surfactants, lubricants, sweeteners, fragrances, preservatives, and the like.

Examples of the quasi-drug composition of the present invention may comprise a disinfectant cleaner, a shower foam, an ointment, a wet tissue, a coating agent, and the like, and the quasi-drug composition of the present invention is preferably prepared in a semi-solid preparation such as an ointment for external use, a lotion, or the like, but the present invention is not limited thereto. Formulation methods, dosages, methods of use, components, and the like of the quasi-drug may be appropriately selected from conventional techniques known in the art.

In the method of brightening the skin, improving wrinkles in the skin, moisturizing the skin, inhibiting bacteria in the skin, and/or preventing hair loss, or promoting hair growth according to the present invention, when a subject is treated with a kakkalide composition, the composition may be in the form of a cosmetic composition, a pharmaceutical composition, or a quasi-drug and/or food composition.

In addition, in the present invention, a food composition for brightening the skin, improving wrinkles in the skin, moisturizing the skin, inhibiting bacteria in the skin, preventing hair loss, or promoting hair growth comprising kakkalide represented by Chemical Formula 1 as an active ingredient is provided.

When the compound of Chemical Formula 1 is provided in the form of a food composition, the composition may comprise auxiliary food additives acceptable in food science in addition to the active ingredient.

The auxiliary food additive refers to an auxiliary component that can be added to food, and is added to manufacture health functional foods of various formulations, and can be appropriately selected and used by those skilled in the art. Examples of auxiliary food additive types comprise various nutritional supplements, vitamins, minerals (electrolytes), flavoring agents such as synthetic flavoring agents and natural flavoring agents, colorants, fillers, pectic acid and a salt thereof, alginic acid and a salt thereof, organic acids, protective colloid thickeners, pH adjusters, stabilizers, preservatives, glycerin, alcohols, carbonating agents used in carbonated beverages, and the like, but the present invention is not limited thereto.

In addition, the food composition may comprise health functional food. In the present invention, “health functional food” refers to a food group or food composition that has added value imparted by a physical, biochemical, or biotechnological method so that the function can be manifested for a specific purpose, and is food designed and processed to sufficiently exert body-controlling functions, such as regulating biological defense rhythm and functions related to disease prevention and recovery, in a living body. The health functional food is more effective in actively maintaining or enhancing health than general food, and health supplement food refers to supplementary food manufactured for the promotion of health. In some cases, terms such as “functional food,” “health food,” and “health supplement food” are used interchangeably.

Specifically, the health functional food is food prepared by adding kakkalide of the present invention to food materials such as beverages, teas, spices, gums, and cookies, or by forming the same into capsules, powders, suspensions, or the like. Although it is suggested that the health functional food has a specific effect on health when ingested, unlike general drugs, since food is used as a raw material, the health functional food has the advantage of having no side effects that may occur in long-term use of drugs.

The food composition may comprise food additives acceptable in food science and may additionally comprise suitable carriers, excipients, and diluents commonly used in the manufacture of health functional foods.

The composition may comprise additional components that are commonly used in food compositions to improve smell, taste, appearance, and the like. For example, vitamins A, C, D, E, B1, B2, B6, B12, niacin, biotin, folate, pantothenic acid, and the like may be comprised. In addition, minerals such as zinc (Zn), iron (Fe), calcium (Ca), magnesium (Mg), manganese (Mn), copper (Cu), and chromium (Cr) may be comprised. In addition, amino acids such as lysine, tryptophan, cysteine, and valine may be comprised. The form that the health functional food of the present invention can take is not limited, and all foods in a conventional sense may be comprised, and the term “health functional food” can be used interchangeably with terms known in the art such as “functional food.”

In addition, depending on the choice of a person skilled in the art, the health functional food of the present invention may be manufactured by adding suitable auxiliary components and well-known additives that can be comprised in food. Examples of food that can be added comprise meat, sausages, bread, chocolate, candies, snacks, cookies, pizza, ramen, miscellaneous noodles, gums, a dairy product such as ice cream, various soups, beverages, teas, drinks, alcoholic beverages, vitamin complexes, and the like, and the health functional food may be manufactured by adding the food to extracts, teas, jellies, juices, and the like produced using kakkalide of the present invention represented by Chemical Formula 1 as a main ingredient. In addition, examples of the health functional food comprise food used as feed for animals.

The composition of the present invention may additionally comprise a brightening component, anti-wrinkle component, moisturizing component, bacteria inhibiting component, hair loss preventing component, or hair growth promoting component known in the art. The additional component may be comprised in an amount of 0.0001 to 10% by weight based on the total weight of the composition, and the content range may be adjusted according to requirements such as skin safety, ease of formulation of kakkalide represented by Chemical Formula 1, or the like.

In the present invention, “effective amount” means an amount of a compound capable of sufficiently exerting the effect of brightening the skin, improving wrinkles in the skin, moisturizing the skin, inhibiting bacteria in the skin, preventing hair loss, or promoting hair growth.

The effective amount of kakkalide represented by Chemical Formula 1 comprised in the composition of the present invention may vary depending on the form in which the composition is commercialized, the method in which kakkalide is applied to the skin, and the time for which kakkalide stays in the skin. For example, when the composition is commercialized as a pharmaceutical product, the composition may comprise a higher concentration of kakkalide represented by Chemical Formula 1 than when the composition is commercialized as a cosmetic product that is routinely applied to the skin. Therefore, the daily dosage is 0.1 to 500 mg/kg in terms of the amount of kakkalide and may be administered once or in divided doses two to six times a day.

In the present invention, when it is stated that a subject is treated with kakkalide of the present invention, it may mean that the treatment comprised orally administering kakkalide or applying kakkalide to the skin.

The numerical values set forth herein should be construed as comprising equivalent ranges unless otherwise specified.

### DETAILED DESCRIPTION OF EXEMPLARY EMBODIMENTS

Hereinafter, the present invention will be described in detail by way of exemplary embodiments. However, the following exemplary embodiments are only illustrative of the present invention, and the contents of the present invention are not limited to the following exemplary embodiments.

### Experimental Example 1

### Brightening Effect According to Reduction in Total Amount of melanin at Cellular Level

In order to evaluate a brightening effect according to inhibition of melanogenesis, an extract was added to a mouse melanoma cell (B-16 mouse melanoma cell) culture, and the total amount of melanin was measured according to a method described in Lotan R. et al. (Cancer Res. 40:3345-3350, 1980). For this experiment, toxicity with respect to the mouse melanoma cells was first evaluated, and the brightening effect was evaluated at a concentration not inducing toxicity. Dimethyl sulfoxide (DMSO) was used as a negative control, and arbutin was used as a positive control.

Specifically, A compound of Formula 1 was added to a medium of each sample so that the final concentration of the compound of Formula 1 is 1 ppm, 10 ppm, 100 ppm, or 500 ppm, and Daidzein was also added to the medium so that the final concentration of Daidzein is 1 ppm, 10 ppm, 100 ppm, or 500 ppm. Arbutin was added to the medium so that the final concentration was 100 ppm. In each of the mediums, melanoma cells were cultured for three days. Subsequently, the cells were treated with trypsin, detached from the culture plate, and centrifuged, and then melanin was extracted. The detached cells were boiled for 10 minutes by adding 1 ml of a sodium hydroxide solution (1 N concentration) so that melanin was dissolved, and by measuring an absorbance at 400 nm using a spectrophotometer, the amount of melanin production was measured.

The amount of melanin was measured by a method of indicating absorbance per unit cell count (1×10⁶ cells), The amount of melanin production in each experimental group with respect to the negative control was calculated as a relative value, and the value obtained by subtracting the amount of melanin production in each experimental group from 100% of the melanin production amount in the negative control group was expressed as the inhibition rate (%). The results are shown in Table 1. The experiment was performed three times for each sample, and averages of measurements were obtained.

TABLE 1

| Sample | Inhibition rate (%) |
|---|---|
| Negative control | 100 |
| Positive control (Arbutin, 100 ppm) | 56.05 |
| Chemical Formula 1 (1 ppm) | 35.29 |
| Chemical Formula 1 (10 ppm) | 48.05 |
| Chemical Formula 1 (100 ppm) | 57.62 |
| Chemical Formula 1 (500 ppm) | 68.42 |
| Daidzein (1 ppm) | 18.73 |
| Daidzein (10 ppm) | 16.45 |
| Daidzein (100 ppm) | 20.76 |
| Daidzein (500 ppm) | 30.22 |

Referring to Table 1, it can be seen that kakkalide represented by Chemical Formula 1 has an excellent melanin reduction effect even at a low concentration as compared to daidzein, which is also a component of Pueraria lobata flowers, and is thus useful for brightening the skin.

### Experimental Example 2

### Effect of Promoting Type I Collagen Synthesis in Human-Derived Fibroblasts

A sample was added to a human-derived fibroblast culture medium to evaluate the effect of promoting type I collagen synthesis at the cellular level. The synthesized collagen was quantified using a Procollagen Type I C-Peptide Enzyme Immunoassay kit (PICP EIA kit). In order to measure the amount of collagen synthesis, a compound of Formula 1 was added to the fibroblast culture medium (Dulbecco's Modified Eagle Medium (DMEM)) so that the final concentration of compound of Formula 1 was 0.1 ppm, 1 ppm, or 10 ppm in each medium, and Genistein was also added to the fibroblast culture medium (Dulbecco's Modified Eagle Medium (DMEM)) so that final concentration of Genistein was 0.1 ppm, 1 ppm, or 10 ppm. After culturing for 48 hours, and then the culture medium was taken, and, by using the PICP EIA kit, the degree of type I collagen synthesis at each concentration was measured at 450 nm using a spectrophotometer.

In order to compare the effect, the culture medium of fibroblasts without sample treatment was used as a negative control, and 10 ng/ml TGF-β was used as a positive control, and the degrees of collagen synthesis therein were measured in the same manner as for testing an added sample. A rate of increase in collagen synthesis was calculated as a ratio of the amount of collagen production relative to that of the negative control, and the results are shown in Table 2. The experiment was performed three times for each sample, and averages of measurements were obtained.

TABLE 2

| | Amount of type I collagen | Increase |
|---|---|---|
| Sample | production (ng/ml) | rate (%) |
| Negative control | 151.4 | — |
| Positive control (TGF-β 10 ng/ml) | 172.35 | 13.84 |
| Chemical Formula 1 (0.1 ppm) | 157.74 | 4.19 |
| Chemical Formula 1 (1 ppm) | 170.41 | 12.56 |
| Chemical Formula 1 (10 ppm) | 175.72 | 16.07 |
| Genistein (0.1 ppm) | 155.24 | 2.53 |
| Genistein (1 ppm) | 158.75 | 4.85 |
| Genistein (10 ppm) | 165.93 | 8.59 |

Referring to Table 2, it can be seen that kakkalide represented by Chemical Formula 1 is more effective in synthesizing collagen than genistein, which is also a component of Pueraria lobata flowers, and thus is useful for improving wrinkles.

### Experimental Example 3

### Testing of Increase in HAS2, HAS3, and AQP3 Expression in Human Keratinocytes

Human keratinocytes (HaCaT) were dispensed into a 6-well cell culture plate at 2×10⁵ cells/well and cultured for 24 hours in a DMEM medium containing 10% fetal bovine serum (FBS). The medium was replaced with a DMEM medium without 10% FBS and calcium (Ca²⁺), and the cultured cells were treated with the compound of Chemical Formula 1, which had been diluted with a medium, for 24 hours. After the 24 hours had passed, the cells were recovered and then washed with a cold phosphate buffer solution (PBS), and total RNA was extracted using an RNeasy mini kit (Qiagen, Germany). A reverse transcription reaction was performed using RNA and a cDNA Synthesis kit (PhileKorea, Korea). The synthesized cDNA was diluted with water after being quantified, and the same amount thereof was used for all reactions. HAS2, HAS3, and AQP3 primers from a TaqMan® Universal Master Mix II and TaqMan® Gene Expression Assays (Thermo Fisher, USA) were used. Real-time quantitative polymerase chain reaction (PCR) was carried out using a StepOnePlus® Real-time PCR System (Applied Biosystems, USA). The experimental results were obtained through calculation performed by a ΔΔCt method based on a housekeeping gene glyceraldehyde-3-phosphate dehydrogenase (GAPDH). The levels of HAS2, HAS3, and AQP3 gene expression in each sample were quantified based on the mRNA expression level (normalized to 1.0) of the negative control to which the sample was not added, and the results are shown in Table 3. As a positive control, 10 ng/ml EGF recombinant human protein (Life Technologies, USA) was used.

TABLE 3

| | Increased | Increased | Increased |
|---|---|---|---|
| | HAS2 mRNA | HAS3 mRNA | AQP3 mRNA |
| Sample | amount | amount | amount |
| Negative control | 1 | 1 | 1 |
| EGF 10 ng/ml | 16.42 | 5.24 | 1.09 |
| Chemical Formula 1 | 2.28 | 3.97 | 1.75 |
| 10 ppm | | | |
| Chemical Formula 1 | 6.64 | 5.23 | 4.17 |
| 100 ppm | | | |
| Genistein (10 ppm) | 1.06 | 2.86 | 1.09 |
| Genistein (100 ppm) | 2.74 | 3.34 | 2.47 |

Referring to Table 3, it can be seen that the compound of Chemical Formula 1 is more effective in expressing hyaluronic acid synthesis than the negative control, considering the higher levels of HAS2, HAS3, and AQP3 expression.

Therefore, it can be seen that the composition containing kakkalide of the present invention represented by Chemical Formula 1 is useful for moisturizing the skin.

### Experimental Example 4

### Effect of Inhibiting Microbes (Effect of Inhibiting Dandruff-Causing Fungi)

This experiment was carried out by a serial dilution method and a method of measuring a minimum inhibitory concentration (MIC), using an MHB2 medium (for bacteria) or a PDB medium (for fungi). The bacteria or fungi were inoculated at about 10⁶/well. Three types of general bacteria, two types of fungi, and Pityrosporum ovale were inoculated and cultured, and the degree of bacterial/fungal growth at each sample concentration was observed to determine the minimum concentration for inhibiting bacterial/fungal growth.

TABLE 4

| | Minimum inhibitory concentration (%) | | | | | |
|---|---|---|---|---|---|---|
| | | | | | | Pityrosporum |
| Sample | E. coli | S. aureus | P. aeruginosa | A. niger | C. albicans | ovale |
| Zinc pyrithione | 0.002 | 0.001 | 0.002 | 0.02 | 0.02 | 0.0002 |
| Chemical | 0.05 | 0.01 | 0.05 | 0.1 | 0.05 | 0.0003 |
| Formula 1 | | | | | | |
| Genistein | 0.1 | 0.03 | 0.07 | 0.2 | 0.08 | 0.0005 |

Referring to Table 4, it can be seen that like zinc pyrithione (positive control) having an excellent antimicrobial effect against Pityrosporum ovale, which is a dandruff-causing fungus, the compound of Chemical Formula 1 has an excellent antimicrobial effect. Therefore, it can be seen that a composition comprising kakkalide of the present invention represented by Chemical Formula 1 is useful for inhibiting bacteria.

### Experimental Example 5

### Investigation of Wnt/β-Catenin Signaling Pathway Activation Effect of Extract

The effect of the compound of Chemical Formula 1 on the activation of the Wnt/β-catenin signaling pathway was investigated using TOPflash (T-cell factor reporter plasmid) Kit (Millipore, USA). Human dermal papilla cells were inoculated into a 24-well plate at 2×10⁵/well, cultured for 24 hours, treated with the compound of Chemical Formula 1 so that the concentration was 10 μg/ml, and then additionally cultured for 15 hours, and firefly luciferase activity was subsequently measured using a dual luciferase assay kit (Promega, USA).

In order to accurately measure the luciferase activity, the experiment was performed three times for each sample, an increase rate (%) was calculated as a ratio of activity relative to that of a control was calculated, and the results are shown in Table 5. The negative control was an untreated group. As a positive control, 6-bromoindirubin-3′-oxime (BIO) was used, and BIO is a material known to inhibit GSK-3 in the Wnt signaling system.

TABLE 5

| Sample | Increase rate (%) |
|---|---|
| Control (not treated) | 100 |
| BIO 1 μM | 153 |
| Chemical Formula 1 (10 μg/ml) | 160 |
| Genistein (10 μg/ml) | 140 |

Referring to Table 5, it can be seen that kakkalide represented by Chemical Formula 1 is more effective in activating the Wnt/β-catenin signaling pathway than the control. Therefore, it can be seen that a composition comprising kakkalide of the present invention represented by Chemical Formula 1 is useful for preventing hair loss or promoting hair growth.

### Experimental Example 6

### Effect of Proliferating Dermal Papilla Cells

This experiment was performed using the following media 1) to 3):

1) DMEM (10% FBS, 1% P/S)

2) Depletion medium: 5% charcoal-stripped, FBS-containing, phenol red-free DMEM

3) Serum-free (SF) medium: DMEM (1% P/S).

On days 0 to 5, the following various experiments were performed to confirm a dermal papilla cell proliferation effect.

Day 0: At 10 a.m., cells were seeded (5,000 cells/well)

Day 1: At 10 a.m., after changing the depletion medium, cells were cultured for 24 hours.

Day 2: At 10 a.m., CCK-8 was measured (average initial value: 0.586) before the cells were treated with a sample, and subsequently, cells were treated with a sample.

Day 5: At 10 a.m., CCK-8 was measured (it is determined that cells do not die, and the initial number is maintained at the of the average control value of 0.556).

TABLE 6

| Sample | Average (ratio) | stdev |
|---|---|---|
| Control (DMEM) | 1.00 | 0.03 |
| Serum-free (SF) | 0.68 | 0.04 |
| Chemical Formula 1 100 ppm | 1.34 | 0.07 |
| Genistein 100 ppm | 1.10 | 0.05 |

stdev: standard deviation

Referring to Table 6, it can be seen that in a group treated with kakkalide of the present invention represented by Chemical Formula 1, the dermal papilla cells proliferated more than in the control.

Therefore, it can be seen that a composition comprising the compound of the present invention represented by Chemical Formula 1 is useful for preventing hair loss.

A composition comprising kakkalide according to the present invention can be variously used for brightening the skin, improving wrinkles in the skin, moisturizing the skin, inhibiting bacteria in the skin, preventing hair loss, or promoting hair growth.

What is claimed is:

1. A method of brightening the skin, comprising treating a subject with kakkalide represented by the following Chemical Formula 1:

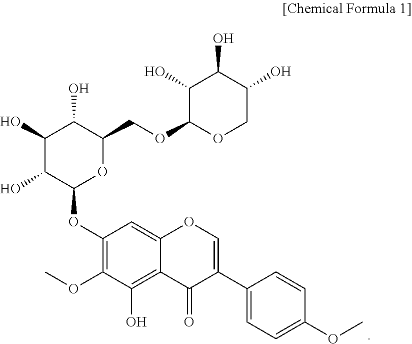 [SMILES: COc1ccc(-c2coc3cc(O[C@@H]4O[C@H](CO[C@@H]5OC[C@@H](O)[C@H](O)[C@H]5O)[C@@H](O)[C@H](O)[C@H]4O)c(OC)c(O)c3c2=O)cc1]

2. The method of claim 1, wherein the kakkalide is comprised in a composition.

3. The method of claim 1, wherein the treating with the kakkalide comprises orally administering the kakkalide or applying the kakkalide to the skin.

4. A method of preventing or treating a disease related to excessive pigmentation, comprising treating a subject with kakkalide represented by the following Chemical Formula 1:

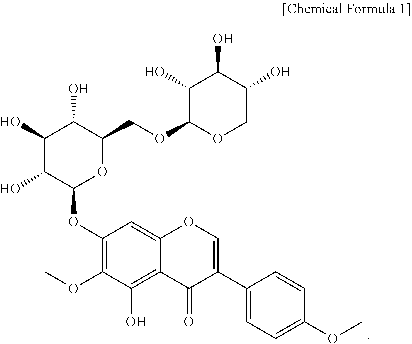 [SMILES: COc1ccc(-c2coc3cc(O[C@@H]4O[C@H](CO[C@@H]5OC[C@@H](O)[C@H](O)[C@H]5O)[C@@H](O)[C@H](O)[C@H]4O)c(OC)c(O)c3c2=O)cc1]

5. The method of claim 4, wherein the disease related to excessive pigmentation is melasma, freckles, or hyperpigmentation.

6. The method of claim 4, wherein the treating with the kakkalide comprises orally administering the kakkalide or applying the kakkalide to the skin.

7. A method of improving wrinkles in the skin, comprising treating a subject with kakkalide represented by the following Chemical Formula 1:

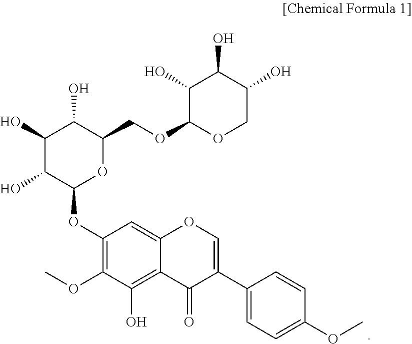 [SMILES: COc1ccc(-c2coc3cc(O[C@@H]4O[C@H](CO[C@@H]5OC[C@@H](O)[C@H](O)[C@H]5O)[C@@H](O)[C@H](O)[C@H]4O)c(OC)c(O)c3c2=O)cc1]

8. The method of claim 7, wherein the treating with the kakkalide comprises orally administering the kakkalide or applying the kakkalide to the skin.

9. The method of claim 7, wherein the treating with the kakkalide promotes type I collagen synthesis and thus improves the wrinkles in the skin.

10. A method of preventing, improving, or treating symptoms of dry skin, comprising treating a subject with kakkalide represented by the following Chemical Formula 1:

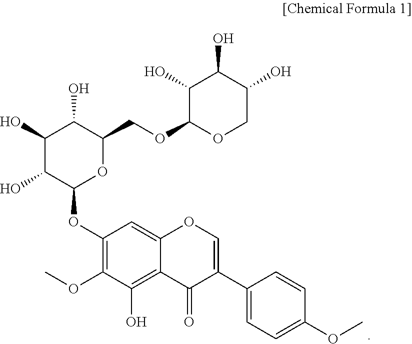 [SMILES: COc1ccc(-c2coc3cc(O[C@@H]4O[C@H](CO[C@@H]5OC[C@@H](O)[C@H](O)[C@H]5O)[C@@H](O)[C@H](O)[C@H]4O)c(OC)c(O)c3c2=O)cc1]

11. The method of claim 10, wherein the treating with the kakkalide comprises orally administering the kakkalide or applying the kakkalide to the skin.

12. The method of claim 10, wherein the symptoms of the dry skin comprise lack of moisture in the skin, atopic dermatitis, psoriasis, xeroderma, eczema, or xeroderma pigmentosum.

13. A method of inhibiting bacteria in the skin, comprising treating a subject with kakkalide represented by the following Chemical Formula 1:

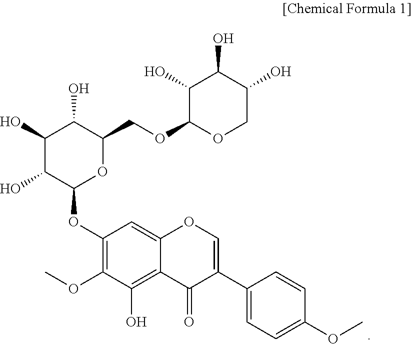 [SMILES: COc1ccc(-c2coc3cc(O[C@@H]4O[C@H](CO[C@@H]5OC[C@@H](O)[C@H](O)[C@H]5O)[C@@H](O)[C@H](O)[C@H]4O)c(OC)c(O)c3c2=O)cc1]

14. The method of claim 13, wherein the treating with the kakkalide comprises orally administering the kakkalide or applying the kakkalide to the skin.

15. A method of preventing or treating dandruff, comprising treating a subject with kakkalide represented by the following Chemical Formula 1:

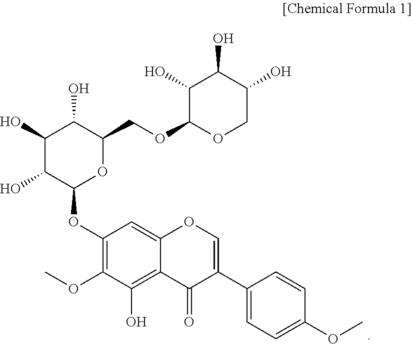 [SMILES: COc1ccc(-c2coc3cc(O[C@@H]4O[C@H](CO[C@@H]5OC[C@@H](O)[C@H](O)[C@H]5O)[C@@H](O)[C@H](O)[C@H]4O)c(OC)c(O)c3c2=O)cc1]

16. The method of claim 15, wherein the kakkalide inhibits growth of Pityrosporum ovale and thus prevents or treats dandruff

17. A method of preventing hair loss or promoting hair growth, comprising treating a subject with kakkalide represented by the following Chemical Formula 1:

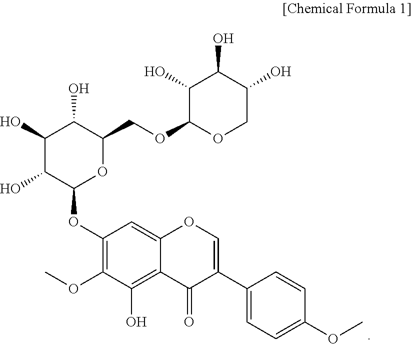 [SMILES: COc1ccc(-c2coc3cc(O[C@@H]4O[C@H](CO[C@@H]5OC[C@@H](O)[C@H](O)[C@H]5O)[C@@H](O)[C@H](O)[C@H]4O)c(OC)c(O)c3c2=O)cc1]

18. The method of claim 17, wherein the treating with the kakkalide comprises orally administering the kakkalide or applying the kakkalide to the skin.

19. The method of claim 17, wherein the kakkalide activates Wnt/β-catenin signaling pathway and/or promotes proliferation of dermal papilla cells, and thus prevents hair loss or promotes hair growth.
